(19) United States
(12) Patent Application Publication
(10) Pub. No.: US 2021/0088238 A1
(43) Pub. Date: Mar. 25, 2021

(54) DUST REMOVAL DEVICE AND DUST REMOVAL SYSTEM

(71) Applicant: DAIKIN INDUSTRIES, LTD., Osaka (JP)

(72) Inventors: Kei Suzumura, Osaka (JP); Tomohiro Yabu, Osaka (JP)

(21) Appl. No.: 17/112,294

(22) Filed: Dec. 4, 2020

Related U.S. Application Data
(63) Continuation of application No. PCT/JP2019/022718, filed on Jun. 7, 2019.

(30) Foreign Application Priority Data
Jun. 8, 2018 (JP) 2018-110176

(51) Int. Cl. F24F 9/00; F24F 13/072

(52) U.S. Cl. CPC F24F 9/00; F24F 2120/12; F24F 2009/007; F24F 13/072

(57) ABSTRACT

A dust removal device blows air toward a subject and removes dust. The dust removal device includes a fan arranged to blow air, and a blow-out location changing part arranged to change a blow-out location of air that is blown by the fan and to move toward the subject. The blow-out location changing part is arranged to change the blow-out location in accordance with a height of the subject such that the air is blown toward the subject from a neck down.

### CROSS-REFERENCE TO RELATED APPLICATIONS

This is a continuation of International Application No. PCT/JP2019/022718 filed on Jun. 7, 2019, which claims priority to Japanese Patent Application No. 2018-110176 filed on Jun. 8, 2018. The entire disclosures of these applications are incorporated by reference herein.

### BACKGROUND

### Field of Invention

The present disclosure relates to a dust removal device and a dust removal system.

### Background Information

There is a known air shower device that blows air with respect to a subject (a person that enters a room) through an air nozzle to beat powder dust out of the subject (refer to, for example, Japanese Patent No. 5464726).

### SUMMARY

A first aspect of the present disclosure is intended for a dust removal device that blows air toward a subject and remove dust. The dust removal device includes a fan that blows air, and a blow-out location changing part that changes a blow-out location of air that is blown by the fan and moves toward the subject. The blow-out location changing part changes the blow-out location in accordance with a height of the subject such that the air is blown toward the subject from the neck down.

### BRIEF DESCRIPTION OF THE DRAWINGS

FIG. 1 is a perspective view of a configuration of a dust removal device according to Embodiment 1.

FIG. 2 is a planar sectional view of a state in which air is blown in a direction from an indoor space to an outdoor space.

FIG. 3 is a side view of a state in which air is blown toward a subject from the neck down in accordance with the height of the subject.

FIG. 4 is a back view of a state in which air is blown to a subject downwardly from above.

FIG. 5 is a side view of a state in which air is blown toward another subject from the neck down in accordance with the height of the other subject.

FIG. 6 is a side view of a configuration in which a blow-out location is changeable by switching the opening and closing operations of an opening-closing damper in a dust removal device according to Modification 1.

FIG. 7 is a side view of a configuration in which a blow-out location is changeable by switching the operation of a plurality of fans in a dust removal device according to Modification 2.

FIG. 8 is a plan view of a configuration of a dust removal system according to Embodiment 2, the dust removal system including a dust removal device for a subject and a dust removal device for an article.

### DETAILED DESCRIPTION OF EMBODIMENT(S)

### Embodiment 1

Embodiment 1 will be described.

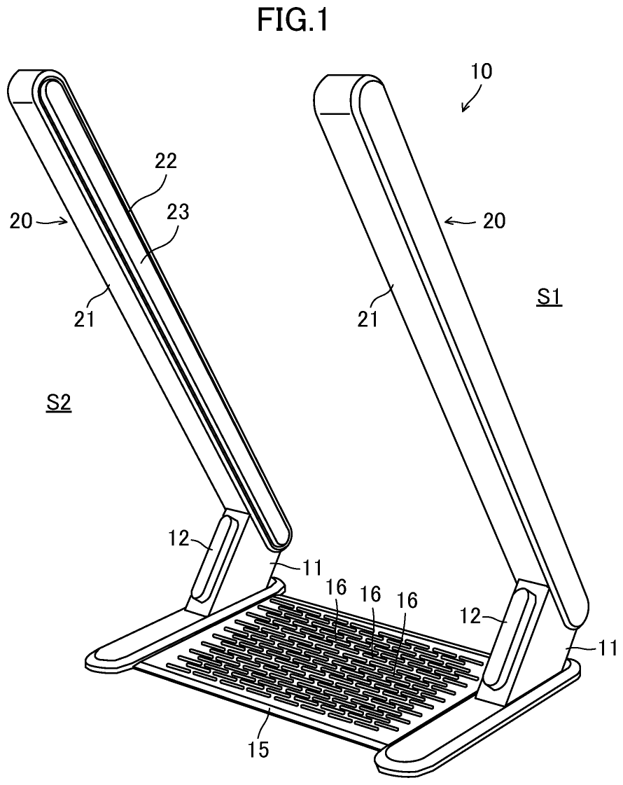

As illustrated in FIG. 1, a dust removal device (10) includes a left-right pair of blower gates (20), left and right support legs (11) that support lower end portions of the blower gates (20), and a floor plate (15) disposed between the left and right support legs (11).

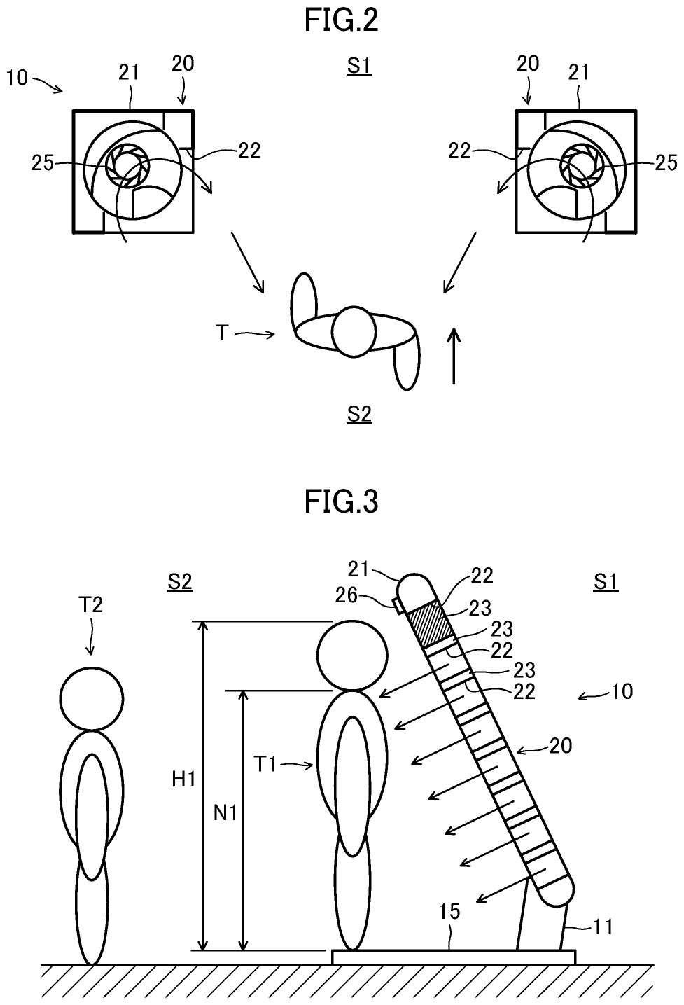

As illustrated in FIG. 2 and FIG. 3, the blower gates (20) each include a gate body (21) with a cross-flow type fan (25) disposed in the inner portion thereof, and a plurality of flaps (23) (blow-out location changing parts) capable of opening and closing the plurality of blow-out ports (22) formed in the gate body (21).

The blower gates (20) are installed at a boundary location between an indoor space (S1) and an outdoor space (S2). The blow-out ports (22) are formed such that air is blown in a lateral direction that crosses an entrance direction in which a subject (T) moves from the outdoor space (S2) toward the indoor space (S1). In other words, the blow-out ports (22) are formed in faces of the left and right blower gates (20), the faces facing each other.

The blower gates (20) each stand in an orientation that is inclined obliquely upward from the indoor space (S1) toward the outdoor space (S2). Consequently, the blow-out ports (22) that open at the near side in the entrance direction are positioned higher than the blow-out ports (22) that open at the far side in the entrance direction.

The support legs (11) support the blower gates (20) so as to be in the above-described inclined orientation. An addition cartridge (12) (an addition part) that adds a useful component to the air blown by the fans (25) is detachably mounted on each of the support legs (11) (refer to FIG. 1).

The useful component contained in the addition cartridge (12) is, for example, a deodorizer, a sterilizing agent, a fragrance agent, and the like. The inner portion of each support leg (11) and the inner portion of the gate body (21) corresponding thereto are in communication with each other. When the fans (25) of the blower gates (20) are driven to rotate, the useful component of the addition cartridges (12) is sent into the inner portions of the gate bodies (21) and blown through the blow-out ports (22) after stirred with air.

A plurality of suction ports (16) are formed at the upper surface of the floor plate (15). The suction ports (16) are in communication with a space of the inner portion of the floor plate (15). In the space of the inner portion of the floor plate (15), a suction mechanism (not illustrated) sucks air.

The dust adhering to the subject (T) is sucked through the suction ports (16) after removed by the air blown from the blower gates (20) toward the subject (T) and is collected in the floor plate (15). Consequently, the dust can be suppressed from floating and adhering again to the subject (T).

When air is blown toward the whole body of the subject (T), the air is also blown onto the face of the subject (T) and has a likelihood of making the subject (T) feel discomfort or disarranging a set hair style thereof.

Thus, in the present embodiment, the blow-out location is changeable in accordance with the height of the subject (T) such that air is blown toward the subject (T) from the neck down.

Specifically, as illustrated in FIG. 3, the blower gates (20) are provided with a detection sensor (26) that detects a height (H1) of a subject (T1) that intends to pass between the blower gates (20). A position (N1) of the neck of the subject (T1) is estimated on the basis of a result of the detection by the detection sensor (26). An image of the whole body of the subject (T1) may be obtained by a camera (not illustrated) or the like, and the position (N1) of the neck of the subject (T1) in the image may be extracted.

The blow-out port (22) in a location where air is to be blown to the face of the subject (T1) is closed by the flaps (23), thereby causing the air to be blown to the subject (T1) from the neck down. In the example illustrated in FIG. 3, among eight blow-out ports (22), the first blow-out port (22) from the top is closed by the flaps (23), which are indicated by hatching.

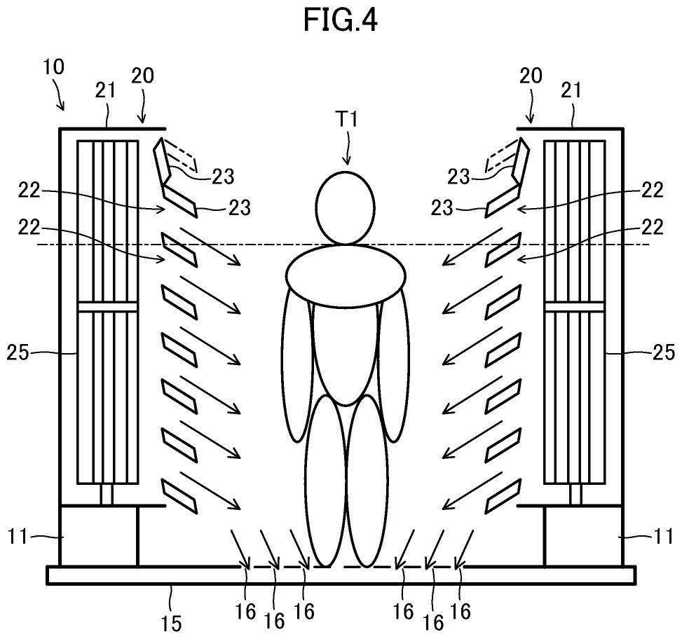

Moreover, as illustrated in FIG. 4, the rotation angle of the flaps (23) is adjusted, thereby causing the air to be blown downwardly from above the subject (T1). Consequently, the dust can be suppressed from floating and adhering again to the subject (T). The removed dust is collected in the inner portion of the floor plate (15) through the suction ports (16) of the floor plate (15).

Meanwhile, dust of pollen and the like largely adheres to the body parts of the subject (T1), and the amount of dust adhering to the parts of the face is small compared with the body parts. Therefore, the influence as a result of the dust that adheres to the parts of the face being taken into the indoor space (S1) is small.

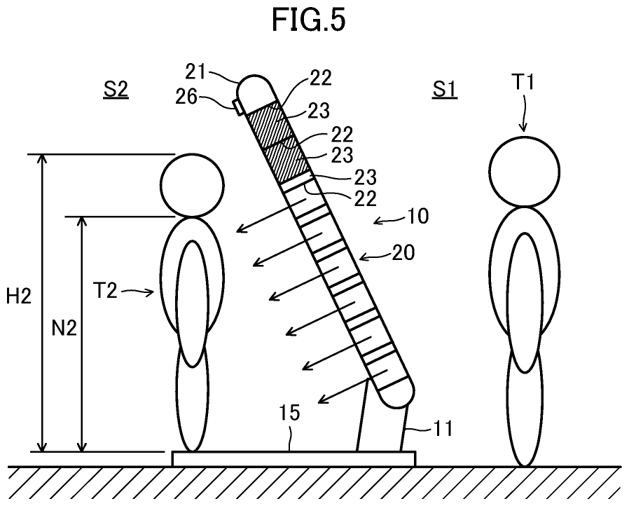

Next, as illustrated in FIG. 5, when a subject (T2) whose height is lower than that of the subject (T1) passes between the blower gates (20), a height (H2) of the subject (T2) is first detected by the detection sensor (26). Then, on the basis of a result of the detection by the detection sensor (26), a position (N2) of the neck of the subject (T2) is estimated.

Then, the blow-out ports (22) in a location where air is to be blown to the face of the subject (T2) are closed by the flaps (23), thereby causing the air to be blown to the subject (T2) from the neck down. In the example illustrated in FIG. 5, among the eight blow-out ports (22), the first and second blow-out ports (22) from the top are closed by the flaps (23), which are indicated by hatching.

### Effects of Embodiment 1

The dust removal device (10) according to the present embodiment blows air toward the subject (T) and removes dust. The dust removal device (10) includes the fans (25) that blow air and the flaps (23) (blow-out location changing parts) that change the blow-out location of the air that is blown by the fans (25) and moves toward the subject (T). The flaps (23) change the blow-out location in accordance with the height of the subject (T) such that air is blown toward the subject (T) from the neck down.

In the present embodiment, the blow-out location of the air is changed in accordance with the height of the subject (T), thereby causing the air to be blown toward the subject (T) from the neck down. Consequently, the air can be suppressed from being blown onto the face of the subject (T).

Further, in the dust removal device (10) according to the present embodiment, the flaps (23) (blow-out location changing parts) blow air toward the subject (T) in a direction from the indoor space (S1) to the outdoor space (S2).

In the present embodiment, air is caused to be blown toward the subject (T) in the direction from the indoor space (S1) to the outdoor space (S2). Consequently, after the dust adhering to the subject (T) is removed, the dust can be discharged along the flow of the air to the outdoor space (S2).

In the dust removal device (10) according to the present embodiment, the flaps (23) (blow-out location changing parts) blow air toward the subject (T) downwardly from above.

In the present embodiment, the air is caused to be blown toward the subject (T) downwardly from above. Consequently, after the dust adhering to the subject (T) is removed, the dust can be suppressed from floating and adhering again to the subject (T).

In the dust removal device (10) according to the present embodiment, the blow-out ports (22) are provided such that air is blown in the lateral direction that crosses the entrance direction in which the subject (T) moves from the outdoor space (S2) toward the indoor space (S1), and the blow-out ports (22) that open at the near side in the entrance direction are positioned higher than the blow-out ports (22) that open at the far side in the entrance direction.

In the present embodiment, the blow-out ports (22) that open at the near side in the entrance direction are positioned higher than the blow-out ports (22) that open at the far side in the entrance direction. Specifically, when the subject (T) moves in the entrance direction, the dust on the upper body of the subject (T) is first removed. Thereafter, even when the removed dust adheres again to the lower body of the subject (T), the dust on the lower body of the subject (T) can be removed by the air blown through the blow-out ports (22) at the far side.

The dust removal device (10) according to the present embodiment also includes the addition cartridges (12) that add the useful component to the air blown by the fans (25).

In the present embodiment, the useful component is added to the air that is blown by the fans (25). Consequently, for example, a deodorizer, a sterilizing agent, a fragrance agent, and the like can be added as the useful component.

### Modification 1 of Embodiment 1

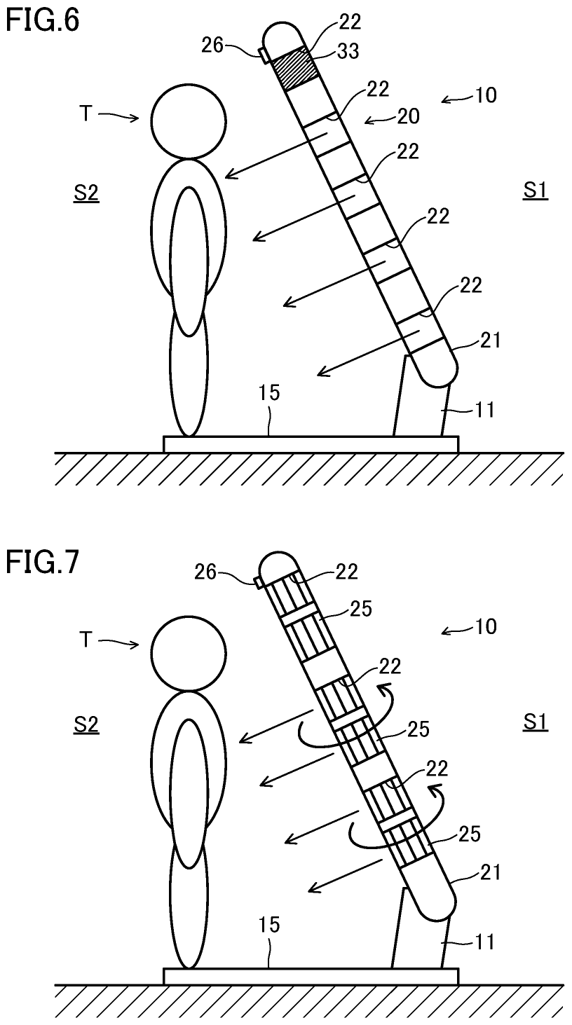

As illustrated in FIG. 6, the gate bodies (21) of the blower gates (20) each have five blow-out ports (22) that open so as to be spaced apart from one another in the vertical direction. The blow-out ports (22) are openable and closable by an opening-closing damper (33) (blow-out location changing part).

When the subject (T) passes between the blower gates (20), the height of the subject (T) is first detected by the detection sensor (26). On the basis of a result of the detection by the detection sensor (26), the position of the neck of the subject (T) is estimated.

The blow-out port (22) in a location where air is to be blown to the face of the subject (T) is closed by the opening-closing damper (33), thereby causing the air to be blown to the subject (T) from the neck down. In the example illustrated in FIG. 6, among the five blow-out ports (22), the first blow-out port (22) from the top is closed by the opening-closing damper (33), which is indicated by hatching.

### Modification 2 of Embodiment 1

As illustrated in FIG. 7, the gate bodies (21) of the blower gates (20) each include three cross-flow type fans (25) disposed so as to be spaced apart from one another in the vertical direction.

When the subject (T) passes between the blower gates (20), the height of the subject (T) is first detected by the detection sensor (26). On the basis of a result of the detection by the detection sensor (26), the position of the neck of the subject (T) is estimated.

The operation of the fan (25) in a location where air is to be blown to the face of the subject (T) is stopped while the remaining fans (25) are operated, thereby causing the air to be blown to the subject (T) from the neck down. In the example illustrated in FIG. 7, among the three fans (25), the operation of the uppermost fan (25) is stopped.

### Embodiment 2

Embodiment 2 will be described.

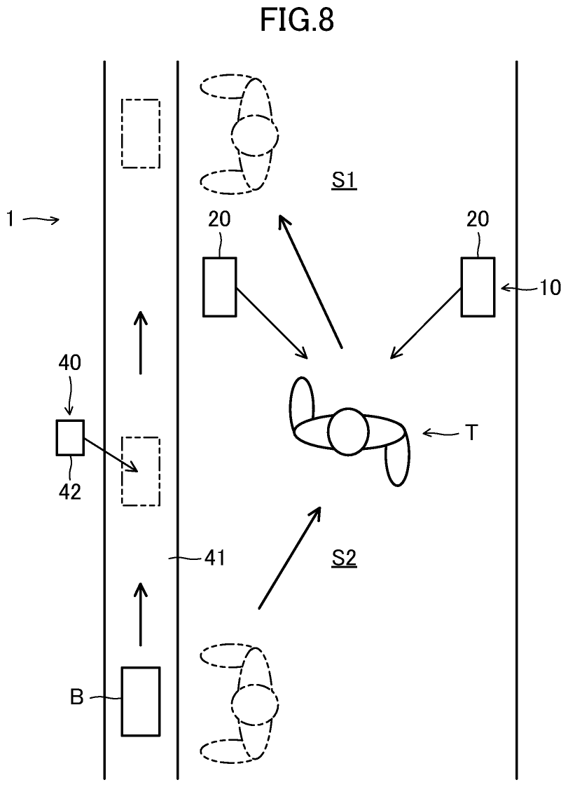

As illustrated in FIG. 8, a dust removal system (1) includes the dust removal device (10) for the subject (T), a conveyance part (41) that conveys an article (B) from the outdoor space (S2) to the indoor space (S1), and a dust removal device (40) for the article (B), such as bags, jackets, and the like.

As in Embodiment 1 mentioned above, the dust removal device (10) for the subject (T) includes the left-right pair of blower gates (20) and can remove the dust adhering to the body of the subject (T) by blowing air toward the subject (T), from the neck below, that passes between the blower gates (20).

The conveyance part (41) is constituted by a belt conveyer that extends from the outdoor space (S2) to the indoor space (S1), a lift that moves in the horizontal direction, and the like. The article (B) placed on the conveyance part (41) is conveyed toward the indoor space (S1).

The dust removal device (40) for the article (B) includes a fan (42) that blows air toward the article (B) during conveyance by the conveyance part (41). The dust removal device (40) for the article (B) is not limited to the fan (42) and may be constituted by, for example, a vibration mechanism that removes dust by applying vibrations to the article (B) during conveyance of the article (B).

In the thus configured dust removal system (1), the subject (T) first places the article (B), such as a hand bag and the like, on the conveyance part (41) and then moves to the dust removal device (10) for the subject (T). The article (B) is conveyed by the conveyance part (41) to the indoor space (S1), and, during the conveyance, dust is removed by the dust removal device (40) for the article (B).

The subject (T) passes through the dust removal device (10), and the dust adhering to the body is thereby removed. Thereafter, the subject (T) collects the article (B) that has been conveyed by the conveyance part (41) and enters the indoor space (S1).

Consequently, the dust adhering to the subject (T) and the dust adhering to the article (B) can be removed by different dust removal devices (10 and 40).

While embodiments and modifications have been described above, it should be understood that various changes in the forms and details are possible without deviating from the gist and the scope of the claims. The above embodiments and the modifications may be combined together or replaced, as appropriate, provided that the functions of objects of the present disclosure are not lost.

As described above, the present disclosure is useful for a dust removal device and a dust removal system.

1. A dust removal device that blows air toward a subject and removes dust, the dust removal device comprising:
a fan arranged to blow air; and
a blow-out location changing part arranged to change a blow-out location of air that is blown by the fan and to move toward the subject,
the blow-out location changing part being arranged to change the blow-out location in accordance with a height of the subject such that the air is blown toward the subject from a neck down.

2. The dust removal device according claim 1, wherein
the blow-out location changing part is arranged to blow air toward the subject in a direction from an indoor space to an outdoor space.

3. The dust removal device according to claim 1, wherein
the blow-out location changing part is arranged to blow air toward the subject downwardly from above.

4. The dust removal device according to claim 1, further comprising:
a plurality of blow-out ports arranged such that air is blown in a lateral direction that crosses an entrance direction in which the subject moves from an outdoor space toward an indoor space, and
the blow-out port that opens at a near side in the entrance direction being positioned higher than the blow-out port that opens at a far side in the entrance direction.

5. The dust removal device according to claim 1, further comprising:
an addition part arranged to add a useful component to air that is blown by the fan.

6. A dust removal system including the dust removal device according to claim 1, the dust removal device being for the subject, the dust removal system further comprising:
an additional dust removal device that is for an article, the additional dust removal device being arranged to blow air toward the article and remove dust.
